(19) United States
(12) Patent Application Publication
(10) Pub. No.: US 2021/0403960 A1
(43) Pub. Date: Dec. 30, 2021

(54) SYSTEMS AND METHODS FOR PRODUCING POLYHYDROXYALKANOATES FROM ORGANIC WASTE

(71) Applicant: THE REGENTS OF THE UNIVERSITY OF CALIFORNIA, Oakland, CA (US)

(72) Inventors: Ruihong Zhang, El Macero, CA (US); Ke Wang, Davis, CA (US)

(73) Assignee: THE REGENTS OF THE UNIVERSITY OF CALIFORNIA, Oakland, CA (US)

(21) Appl. No.: 17/362,562

(22) Filed: Jun. 29, 2021

Related U.S. Application Data
(60) Provisional application No. 63/045,625, filed on Jun. 29, 2020.

(51) Int. Cl. C12P 7/62

(52) U.S. Cl. CPC C12P 7/625

(57) ABSTRACT

Systems and methods are provided for cost effective biosynthesis of polyhydroxyalkanoates (PHA) that have desirable material properties similar to petrochemically derived plastics. Synthesis takes place intracellularly in extreme halophiles grown in saline conditions that selectively reduces contamination from other microbes. The industrial scale PHA production systems use low-cost organic waste feedstocks, spent medium treatment and recycling and enzyme recovery and reuse for efficiency and reduced cost compared to existing processes.

### CROSS-REFERENCE TO RELATED APPLICATIONS

This application claims priority to, and the benefit of, U.S. provisional patent application Ser. No. 63/045,625 filed on Jun. 29, 2020, incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to plastic polymer synthesis systems and methods, and more particularly to an integrated system and method for producing polyhydroxyalkanoates (PHA) biodegradable polymers from organic waste feedstocks with halophilic microorganisms that produce PHA efficiently and at lower cost compared to existing synthetic pathways.

### 2. Background

Plastics have been used as alternative materials to glass or metals because of their durability, moldability and low cost. However, plastics are slow to degrade or decompose over time. The inability of plastics to timely decompose creates long term accumulations in landfills as well as persistent environment pollution in rivers, lakes and oceans. Disposal of petrochemical based plastics by incineration also produces a range of environmentally harmful substances. Environmental concerns over increasingly larger accumulations of plastics landfills and waterways have intensified the search for alternatives.

Biologically synthesized polymers that can degrade aerobically and anaerobically are enticing alternatives to their petrochemical counterparts. Polyhydroxyalkanoates (PHA) are a family of biodegradable plastics that can be used as an environmentally friendly alternative for conventional plastics in various applications.

PHA is a group of 3-hydroxy fatty acids polyesters derived naturally from various types of microbes. It has thermoplastic properties and ecological characteristics, such as renewable origins and is biodegradable in the natural environment. Consequently, PHA has been an emerging bioplastic material in recent years and has become a popular alternative for conventional petroleum-based plastics to protect the environment from greenhouse gas emissions and harmful plastic waste.

PHA has a well-established commercial market and has been made into various products, including packaging films, plastic containers, medical implant materials, drug carriers, nutritional supplements and biofuels, etc. However, success in the marketplace of synthetic biodegradable polymers has been limited by high production costs. For example, the production cost of PHA is usually 3 to 4 times higher than that of petroleum-based plastic resins presenting a major obstacle to their wider use. Reducing the production costs has been a bottleneck for the market expansion of PHA. The cost of the feedstock is one of the main contributors and accounts for over 40% of the total annual operating costs of current systems.

A number of bacteria have been shown to synthesize intracellular PHA, generally in the form of PHA granules. These bacteria include representatives from Alcaligenes, Azotobacter, Chromobacterium, Pseudomonas, Rhodococcus and Eschericha coli.

One aspect of PHA production systems in the art that makes PHA production expensive over those of conventional petrochemical-based plastics is the cost of large amounts of growth media, chemical reagents, enzymes and the energy used in fermentation plants at an industrial scale. Another aspect is the cost of inefficient recovery of the produced PHA from these microorganisms at the end of the process. Accordingly, there is a need for efficient synthesis and recovery processes to allow cost effective production of commercially significant levels of PHA.

### BRIEF SUMMARY

An integrated system and methods for producing polyhydroxyalkanoates (PHA) are provided that permit production costs that are favorable as compared with the production costs of petrochemical-based plastics. Reduced production costs for synthetic biodegradable polymers will make them economically competitive with conventional petrochemical based plastics and overcome a major obstacle to their wider use.

The systems and methods reduce the costs of production of commercially significant levels of PHA, in part, through the use of low-cost organic waste feedstocks, extreme halophiles, reduced energy and chemical consumption, spent growth medium recycling, and enzyme recovery and reuse. These features can be optimized for efficiency and cost and the system can be adapted to a variety of industrial scales.

Since the overall PHA production costs are found to be sensitive to the cost of the initial feedstock, low-cost organic waste sources are used. PHA can be produced through microbial fermentation of renewable feedstocks, including low-cost by-products and waste streams from food processing plants. Organic waste refers to all kinds of biodegradable organic residues. Suitable organic waste includes, but is not limited to, food waste, agricultural residues, organic portion of municipal solid waste, and green waste. The production of biodegradable thermoplastics from organic wastes can also provide many benefits to the environment and allow sustainable development.

In one embodiment, the system and method employ halophilic microorganisms that produce PHA efficiently from volatile fatty acids, sugars, and other nutrients derived from organic waste. In one embodiment, a high salinity environment is created to grow the halophilic microbes with salt and nutrient recycling.

Haloferax mediterranei is an extreme halophilic archaeon that can maintain a robust and pure microbial culture in unsterile conditions. It has been noted for its capability of producing Poly(3-hydroxybutyrate-co-3-hydroxyvalerate) (PHBV), a type of high-quality PHA, from various waste feedstocks, including ethanol stillage, molasses wastewater, cheese whey hydrolysates, olive mill wastewater, macroalgal hydrolysates and fermented food waste. The Haloferax bacteria is used to illustrate the preferred embodiment of the technology. However, it will be understood that other suitable microorganisms can also be used with the systems and methods and produce desired outputs.

The biorefinery system involving this strain as the PHBV producer has several benefits over other freshwater microbes, which include: (1) a cheaper feedstock cost due to the use of waste/byproduct streams; (2) less energy consumption as sterilization and/or pasteurization is unnecessary; (3) less downstream process inputs due to the extraction process facilitated by osmotic shock that is preferably used; (4) recovered digestion enzymes; and (5) recycled and treated saline growth media.

In addition, the process design of the production system is different from the systems using freshwater PHA producers and mixed cultures, mainly in terms of fermentation, downstream PHA extraction and purification processes, as well as the saline media wastewater treatment and recycling.

In one embodiment, the system and method employ one or more steps comprising:

(a) an organic waste decomposition step where organic waste is converted into soluble nutrients, e.g. volatile fatty acids (VFA), sugars, nitrogen and phosphorous that is achieved, for example, by either 1) anaerobic fermentation or 2) hydrolysis by thermal, chemical and enzymatic treatments;

(b) a solid liquid separation step to obtain a particle-free aqueous solution containing the soluble nutrients;

(c) a nutrient concentration step to remove most of water and obtain a highly concentrated nutrient solution;

(d) a PHA production step where the nutrient concentrates are utilized by selected halophilic microorganisms to synthesize PHA intracellularly;

(e) a cell biomass harvesting step which yields cell biomass and recycles saline nutrient solution back to the PHA production step; and

(f) a PHA extraction step where PHA is extracted from cell biomass and becomes the final product from the process.

This system and method provide an efficient vehicle for utilizing organic waste as feedstock for PHA production with high product yield. This technology has high commercial value for converting various organic wastes into high value PHA. Compared to existing PHA production methods, the technology presented herein is expected to be industrially scalable, more energy efficient and less costly than existing systems.

Potential advantages of the technology presented herein over other production systems on PHA include:

(1) The use of organic waste as feedstock not only reduces the quantity of waste goes into landfills or the natural environment, but also reduces the feedstock cost of PHA production.

(2) The use of halophilic microorganisms for PHA production provides robust processes that are resistant to the contamination of other microbes introduced by organic waste sources and the natural environment, making this new system more efficient and less costly than existing PHA production processes.

(3) The use of halophilic microorganisms also offers a much simpler and less expensive method for PHA polymer extraction from cell biomass with higher PHA yield, compared to existing technologies that use energy and chemically intensive methods for extracting the PHA from freshwater microbial species.

According to one aspect of the technology, cost effective systems and methods are provided for producing PHA, a family of biodegradable plastics used as an environmentally friendly alternative for conventional plastics in various applications.

Another aspect of the technology is to provide a method for the production of PHA through microbial fermentation of renewable feedstocks, including low-cost byproducts and waste streams from food processing plants and similar sources.

A further aspect of the technology is to provide systems and methods that are efficient and environment-friendly, producing higher yields and lower waste and media use as compared to existing manufacturing processes.

Another aspect of the technology is to provide an industrial scale PHA production system that is low cost and economically competitive with plastics typically produced from petrochemical sources.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a functional block diagram of a system for low-cost production of biodegradable PHA biopolymers that have desirable material properties similar to petrochemically derived plastics according to one embodiment of the technology.

FIG. 2 is a functional block diagram of a method for producing PHA biopolymers through halophilic microbial fermentation using renewable feedstocks of organic waste according to one embodiment of the technology.

FIG. 3 is a functional block diagram of a system for low-cost production of biodegradable PHA biopolymers from organic waste feedstocks with enzyme, water, and saline media recycling according to one embodiment of the technology.

### DETAILED DESCRIPTION

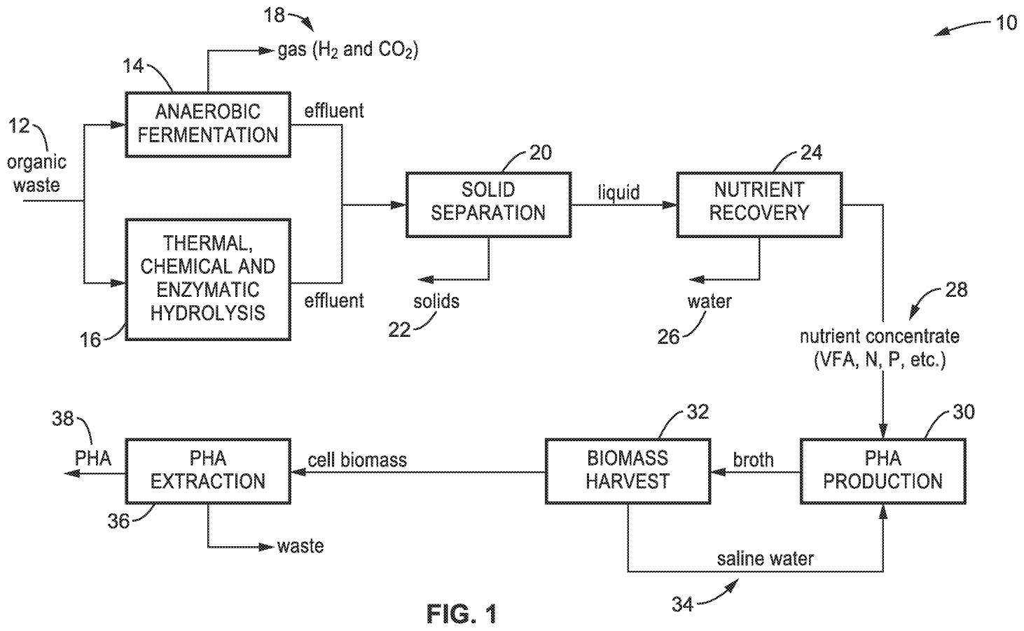

Referring more specifically to the drawings, for illustrative purposes, systems, and methods for industrial scale production of biodegradable plastics with competitive production costs are generally shown. Several embodiments of the technology are described generally in FIG. 1 to FIG. 3 to illustrate the characteristics and functionality of the apparatus, systems, and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, an embodiment of a system 10 for producing polyhydroxyalkanoates (PHA) and related highly valuable biodegradable polyesters from organic waste is shown schematically. The input of the system is organic waste 12 that may be loaded into a reactor 14 for anaerobic fermentation or into a reactor 16 for thermal, chemical or enzymatic hydrolysis. In one embodiment, both types of reactors 14,16 are used in parallel. Organic waste 12 that is used for the input refers to all kinds of biodegradable organic residues. The organic waste 12 may include, but is not limited to, food waste, agricultural residues, organic municipal solid waste, green waste, and food processing waste and by-product streams.

The effluents from either the anaerobic fermentation reactor 14 or the hydrolysis by the thermal, chemical and enzymatic treatment reactor 16 decompose the organic waste 12 into soluble nutrients, e.g. volatile fatty acids (VFA), sugars, nitrogen, phosphorous and micronutrients. The organic waste decomposition step in the anaerobic fermentation reactor 14 may also produce off-gases such as hydrogen and carbon dioxide that may be collected and used a feed stock for other commercially relevant reactions or separated and refined providing added output value to the system.

Reactor effluents that are achieved by either anaerobic fermentation or hydrolysis by thermal, chemical and enzymatic treatments are then placed in a separator 20, either separately or collectively, to perform a solid/liquid separation step. The separator 20 produces a particle-free aqueous nutrient solution by removing suspended solids 22 from the effluents.

The separated liquids produced by the solid separator 20 are processed by a nutrient recovery step in a recovery reactor 24 which removes most of water 26 and obtains a highly concentrated nutrient solution of volatile fatty acids (VFA), sugars, nitrogen and phosphorous 28 for use in the PHA production step. The concentration of the recovered nutrients 28 can be controlled by the amount of water 26 that is extracted by the recovery reactor 24.

PHA production in the system 10 takes place in a PHA production reactor 30. Although one production reactor is referenced for clarity, each unit operation can involve more than one production reactor with staggered input and output streams.

The concentrated nutrient solution input to the production reactor 30 is utilized by halophilic microorganisms to synthesize PHA intracellularly in the reactor 30. Preferred halophilic microorganisms are adapted to a saline water environment for growth. The use of halophilic microorganisms and saline conditions for PHA production provides processes that are resistant to contamination by other microbes that may be introduced by the use of organic waste sources or the natural environment.

The production reactor 30 also provides suitable temperature conditions and other essential conditions for optimum halophilic microorganism growth. After a period of time for growth, the cell biomass from production reactor 30 is harvested from the growth solution from the production reactor in harvester 32. The growth media that has been separated from the biomass in harvester 32 can be filtered and the saline-nutrient media 34 is preferably recycled back to the PHA production reactor 30 for further use making this embodiment of the system more efficient and less costly than existing PHA production processes. In one embodiment, the recycled saline media 34 is further treated with oxidizing chemical such as H₂O₂ or NaClO to improve the quality of the recycled media by removing or deactivating inhibitory compounds.

The PHA is extracted from the harvested biomass in PHA extractor 36 and refined or purified as necessary to produce the final PHA product 38. In one embodiment, the PHA is extracted by changing the osmolarity of the solution containing the harvested cell biomass in PHA extractor 36 leading to cell lysis. The harvested PHA can also be washed and dried into a powder.

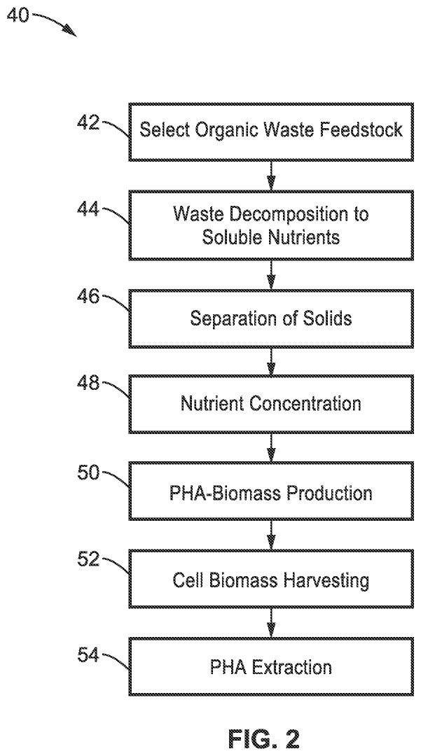

One embodiment of the PHA production methods are illustrated schematically in FIG. 2. Initially, a feedstock of organic waste is selected at block 42 of FIG. 2. The selection at block 42 is influenced by the cost and availability of a constant source of one or more organic waste or waste streams. The overall cost of PHA production was found to be substantially influenced by the cost of the organic waste feedstock. The methods in this embodiment can be illustrated with low-cost by-products and waste streams from food processing plants producing cheese.

For example, the organic waste that is selected at block 42 can be whey permeate, lactose powder, and delactosed permeate (DLP) that are byproduct streams derived from cheese, whey, and lactose manufacturing processes. Whey permeate, for example, is the side stream from the separation and concentration of whey protein. Lactose powder is produced from crystalizing the sugars from whey permeate, leaving behind DLP after the recovery of lactose crystals. These byproduct streams are produced in large quantities from cheese-making facilities and contain rich nutrients including sugars, protein, amino acids, minerals, and micronutrients. However, whey permeates and DLP materials are currently sold as low value products that are used in materials including animal feed, fertilizers, field spread. Converting cheese processing byproducts to PHA can create an additional revenue for dairy processors, and potentially reduce high production costs that can make PHA more competitive in the bioplastic market.

The production system of PHA that is adapted to use cheese processing byproducts may also involve several essential steps, including upstream feedstock pretreatment, fermentation for PHA production, and downstream processes for PHA extraction, purification, and drying. Several economic aspects involved in these processes, including direct capital cost, annual operating cost, revenue, etc. may be relevant to the selection of the feedstock at block 42 of FIG. 2. The substrate cost varies among different types of cheese processing byproducts, and the price of the same type of material can also fluctuate depending on the current market conditions.

Once the waste feedstock is selected and obtained at block 42, the organic waste input is decomposed into soluble nutrients at block 44. The decomposition is preferably accomplished by either anaerobic fermentation or by hydrolysis by thermal, chemical and enzymatic treatments or both. The conditions of fermentation or hydrolysis will be determined by the nature of the organic waste that is selected at block 42.

The purpose of anaerobic fermentation at block 44 is to convert organic waste into VFAs and other nutrients, including nitrogen, phosphorus, potassium, trace elements and micronutrients. The microorganisms that are preferably used for decomposition are hydrolytic and acidogenic microbes, mainly bacteria. As an example of lab-scale anaerobic fermentation, 1-L batch reactors may be used with a working volume of 800 mL. The organic waste and bio-active inoculum are loaded according to the designed VS loading and F/M ratio. For effective VFA production, the VS loading may be in the range of 10 to 40 g VS/L and the F/M ratio may be from 4 to 10 in this illustration. Tap water may be used to fill up to a working volume. As a critical indicator of effectiveness, pH of fermentation broth is normally measured frequently during fermentation. To ensure an anaerobic environment, the headspace of each reactor is normally purged with argon gas for 5 mins to eliminate air. After purging, the headspace can be connected to a 3-L Tedlar bag to collect gases produced during fermentation. The batch reactors may be incubated at about 38° C. for up to four weeks until VFA level stabilizes in one embodiment. Table 1 lists the nutrient levels of fermentation broth resulted from food waste in this illustration.

Solids are separated from the liquids of the fermentation broth or hydrolysis at block 46 and the separated solids are typically discarded. The solid-liquid separation step at block 46 produces an essentially particle-free aqueous solution containing suitable nutrients. The remaining solution preferably undergoes a nutrient concentration step to remove most of water and obtain a highly concentrated nutrient solution at block 48. Optionally, the water from the concentration step at block 48 can be recycled back to the initial input waste processing steps 44, 46.

The PHA production step takes place at block 50 under suitable growth conditions for the microorganism that is selected. Preferred microorganisms are halophilic bacteria in the genus Haloferax adapted to a growth environment in the presence of salts. Although Haloferax bacteria are preferred, other microorganisms such as Halomonas, Haloarcula, Halococcus, Halobacterium and Natrinema genera can be used for PHA biomass production at block 50. Of these microorganisms, Haloferax mediterranei, Haloferax volcanii, Haloferax gibbonsii, Halomonas boliviensis, Halomonas halophila and Halomonas bluephagenesis are particularly preferred.

Halophiles are microorganisms that require a high salinity environment to survive. Halophilic microbes can grow at NaCl salt concentrations from 5% up to 30% (w/v), depending on different species. This high tolerance to salt provides a natural prevention to contamination by other microorganisms that may be present in the media or nutrients. Some halophiles, like Haloferax mediterranei, can synthesize and accumulate PHA granules intracellularly under certain nutrient conditions including using volatile fatty acids (VFA) and sugars as the sole carbon source.

The nutrient concentrates can be used to facilitate growth of the halophilic bacteria strain or strains that are selected for PHA production at block 50. The nutrient concentrates are utilized by the halophilic microorganisms to synthesize PHA intracellularly. Compared to existing technologies that use energy and chemically intensive methods for extracting the PHA from freshwater microbial species, the use of halophilic microorganisms offers a simpler and less expensive method for PHA polymer extraction from cell biomass and with a higher PHA yield.

Suitable PHA production conditions in a laboratory setting can be illustrated with Haloferax mediterranei (ATCC 33500) as an example of PHA-producing halophiles. The aqueous saline medium in this illustration contains mixed salts (NaCl, 156 g/L; MgCl₂.6H₂O, 13 g/L; MgSO₄.7H₂O, 20 g/L; CaCl₂.6H₂O, 1 g/L; KCl, 4 g/L; NaBr, 0.5 g/L and FeCl₃, 5 mg/L) and a trace element solution (ZnSO₄.7H₂O, 100 mg/L; MnCl₂.4H₂O, 30 mg/L; H₃BO₃, 300 mg/L; CoCl₂.6H₂O, 200 mg/L; CuCl₂.2H₂O, 10 mg/L; NiCl₂.6H₂O, 20 mg/L; Na₂MoO₄.H₂O, 30 mg/L) and can be used as a base culturing medium. About 30 mM NaHCO₃ can be used as buffer to maintain a pH 7 during cell cultivation. A 4 g/L VFA mixture containing acetic acid, propionic acid and butyric acid may be used as a carbon source and 3.06 g/L NH₄Cl and 0.5 g/L KH₂PO₄ can be used as nitrogen and phosphorous sources. The strain may be cultured in the liquid medium at 38° C. with 100 mL/min moist aeration until the growth reaches stationary phase. It typically takes around 4 to 7 days for the strain to reach stationary phase, with a maximum PHA yield of around 0.3 g PHA/g VFA. The PHA content is over 50% of cell dry mass.

The grown microorganism cells are separated from the growth media after a sufficient period of time for PHA synthesis at block 52. Centrifugation may be used to harvest cell biomass from the fermentation broth at block 52. The PHA is then extracted from the harvested biomass at block 54. The saline-nutrient media can be recycled back to the PHA production step 50 for further use at block 52.

There are inputs of the system that can be recycled to improve the economics of production, including water, saline water, media with unused nutrients and enzymes. For example, since H. mediterranei cannot use lactose directly, it is necessary to hydrolyze lactose into its monosaccharide constituents, glucose and galactose, before the PHA production. Enzymatic hydrolysis is an environmentally friendly approach, which does not require as much mass and energy inputs as found with acid-catalyzed hydrolysis. The cost of the enzyme may also be high depending on its price and usage in the hydrolysis process. However, the cost can be saved through enzyme reuse or immobilization, which are viable steps in the lactose hydrolysis process.

The cultivation of H. mediterranei may also require media with around 18% total salts to maintain a suitable growth environment. The massive salt input involved in the fermentation step is another important factor which may influence the overall economics of the system. The recycling of the spent salts can reduce the costs of salts as well as the cost of high-saline wastewater treatment.

Finally, PHA is extracted from the harvested biomass and recovered at block 54 of FIG. 2. One advantage of using microorganisms with high salt tolerance is that the extraction of PHA granules from cells is comparatively simple by adding salt-free water to enable cell lysis for extraction at block 54. Accordingly, the harvested cell biomass is then placed in salt-free solution to lyse the cells and release the intracellular PHA polymers. Therefore, it can be seen that the use of halophiles saves energy and chemicals for operational pasteurization, sterilization and downstream PHA extraction.

The technology described herein may be better understood with reference to the accompanying examples, which are intended for purposes of illustration only and should not be construed as in any sense limiting the scope of the technology described herein as defined in the claims appended hereto.

### Example 1

To demonstrate the capabilities of the systems and methods, an industrial scale PHA production system using cheese whey by-product streams as a carbon source was modeled and evaluated. Three types of by-products, (lactose powder, whey permeate, and DLP) were utilized as the carbon source in the models. The cheese processing by-product feedstock was simplified as 168.7 MT/day of lactose, which was a feasible scale for a local cheese processing facility.

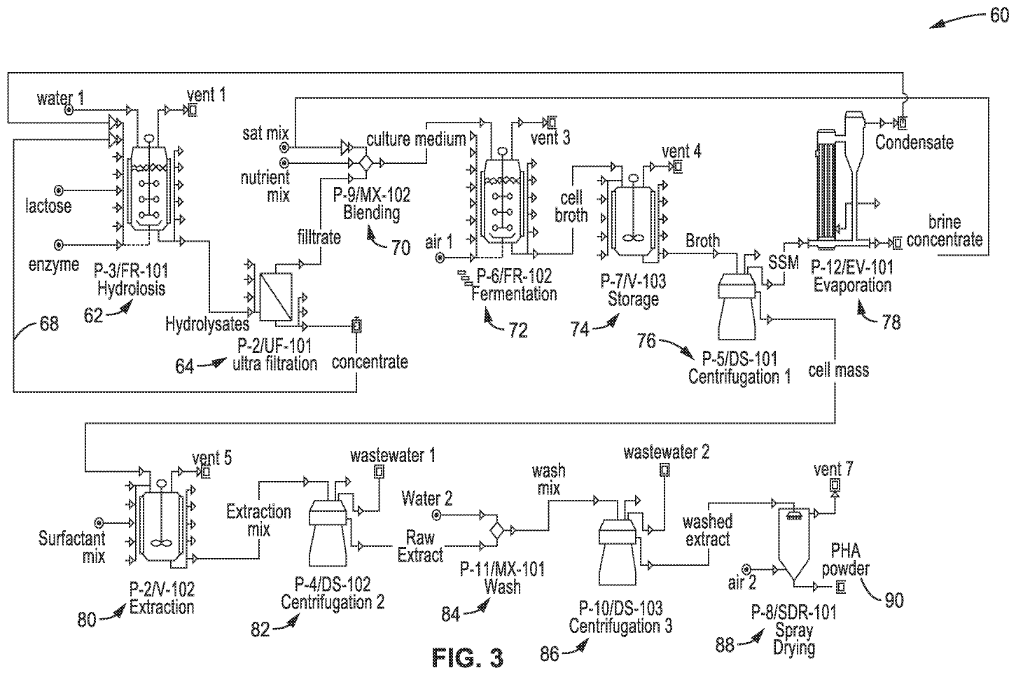

The system structure and flowsheet of one model is shown schematically in FIG. 3. The generic flowsheet of the production system 60 has 12 essential unit operations, which are operated to convert the three types of cheese whey by-product streams into dry PHA powder as the target final product.

The embodiment of the system 60 shown in FIG. 3 is configured for both enzyme reuse and spent salt recycling. The essential unit operations involved in the model were (in the order of process flows): hydrolysis, ultrafiltration, blending, fermentation, storage, evaporation, centrifugation 1, extraction, centrifugation 2, wash, centrifugation 3, and spray drying. Depending on the process design, the additional unit operations of ultrafiltration (after hydrolysis), and evaporation (after centrifugation 1) are optional and may be eliminated. Each of these unit operations is described in greater detail below.

Generally, the process flow of the system 60 shown in FIG. 3 begins with the lactose in the by-product streams being hydrolyzed into glucose and galactose with appropriate amounts of lactase enzyme in the hydrolysis tank. The hydrolysate streams were then mixed with salts and other nutrients in a blending process and fed into a fermentation tank where the microbe H. mediterranei was inoculated to produce PHA polymers intracellularly over time. The fermentation was designed to be operated in a staggered mode with 5 fermenters with a retention time of 5 days. At the end of fermentation, the cell broth was transferred into a storage tank and further processed through centrifugation to separate the cells from the spent medium. The cells were then subjected to an extraction process via water addition that caused osmotic shock and cell lysis to release PHA polymers. The PHA was then processed through consecutive runs of centrifugation and washing to improve purity. The washed PHA was finally processed with a spray dryer which yielded a dry PHA powder with less than a 5% moisture content (MC).

Referring again to FIG. 3, the first unit 62 is hydrolysis. The hydrolysis process in this embodiment starts with a hydrolysis tank as the first unit operation. The hydrolysis tank of the hydrolysis unit 62 has inputs for feedstock, enzymes and water. During this process, the lactose of the feedstock was broken down into monosaccharides through enzymatic hydrolysis. The input streams of this unit operation were: (1) lactose, 168.7 MT/day; (2) water, 744.7 MT/day (calculated based on the density of a solution with 20% lactose; and (3) lactase enzyme, 0.53 MT/day. The operational conditions of hydrolysis were set at Charge 1: water, 2500 gal/min, 79.1 min; Charge 2: lactose, 560 MT/h, 20 min; and Charge 3: enzyme, 15 min; agitation: 12 h; heating: 37° C., 12 h; hydrolysis: 12 h, 80% maximum working volume, 95% reaction extent. The stoichiometric equation of hydrolysis (with mass coefficients) was:

$$342.30 lactose + 18.02 water {\to }^{lactose} 180.16 glucose + 180.16 glactose .$$

Right after the reaction, 100% vessel volume was transferred out with a flow rate of 2500 gal/min for 96.2 min. The compounds of output stream (hydrolysates) were water, 736.3 MT/batch; glucose, 84.4 MT/batch; galactose, 84.4 MT/batch; lactose, 8.4 MT/batch; and enzyme, 0.53 MT/batch.

Optionally, the hydrolysates from the hydrolysis unit 62 can be directed through an ultrafiltration unit 64 that facilitates enzyme reuse. In the ultrafiltration process, it was assumed that 100% enzyme was rejected (rejection factor=1) and the permeate stream was 80% (v/v) of feed stream (concentration factor=5). The duration of the process was set to be 8 hours per batch. The membranes used in this process were DFT membranes with a pore size of 0.45 micron, and the replacement frequency of the membranes was once per every 5000 operating hours.

The filtrate from the process containing hydrolysates went to the next fermentation process for cell cultivation and PHBV production. The concentrate stream 68 containing mostly spent enzyme went back to the next hydrolysis batch run in unit 62, where 80% of spent enzyme with 20% fresh enzyme were used.

Following the hydrolysis step and optional ultrafiltration step, the hydrolysates stream was then blended with the salts and nutrient mix at blending unit 70 before feeding into the second fermentation tank in the fermentation unit 72. A mixed salt stock named minimum saline medium (MSM) was registered with the composition as follows: NaCl, 156 g/L; MgCl₂.6H₂O, 13 g/L; MgSO₄.7H₂O, 20 g/L; CaCl₂.6H₂O, 1 g/L; KCl, 4 g/L; NaBr, 0.5 g/L and FeCl₃, 5 mg/L. The salinity of MSM was measured to be 18.8 parts per thousand (ppt), producing a saltwater density of around 1.13 kg/L.

The mass input of MSM was calculated as 172 MT/batch based on this density. The hydrolysate from the hydrolysate unit 62 operation was used as the carbon source for the fermentation in the fermentation unit 72. An additional nutrient mix including mostly 16.9 MT/batch of ammonium chloride was used to provide essential nutrients in addition to the carbon source feed.

The fermentation unit 72 process was the main operation of the system, where H. mediterranei was cultured for PHBV production using the hydrolysates derived from cheese whey by-product streams. The input streams of fermentation were culture medium coming out of the previous blending process 70, which consisted of cheese whey hydrolysates, MSM, nutrient mix, etc. Forced aeration was provided with a flow rate of 1 volume per volume per minute (vvm). There were 5 identical fermentation tanks operated in a staggered batch mode in the system. One tank was filled with culture medium and started the fermentation each day, while another tank was emptied and cleaned to prepare for the next day fermentation. The fermentation time for each batch was assumed to be 5 days. Each batch fermentation adopted the same operational conditions: continuous agitation, heating (37° C.), and venting.

The PHBV yield was assumed as 0.2 g/g sugar, which number was obtained from prior experiments. The PHBV content was assumed to be around 60% cell dry mass (CDM). Both reactions were set to have a 95% conversion efficiency.

After fermentation, 90% of cell broth was transferred out for later use in downstream processes for PHBV production. Meanwhile, the remaining 10% of the cell broth was left in the fermentation tank to be used as the seed for the subsequent batch productions. The output streams in the cell broth were: PHBV (contained in the cells), 31.8 MT/batch; residual biomass, 20.1 MT/batch; MSM, 155.1 MT/batch; glucose, 3.8 MT/batch; galactose, 3.8 MT/batch; lactose, 7.6 MT/batch; ammonium chloride, 0.7 MT/batch; enzyme, 0.5 MT/batch; water, 691.5 MT/batch. After each fermentation batch, the harvested cell broth from the fermentation unit 70 was temporarily stored in a storage tank of storage unit 74 for further processing downstream.

In the embodiment shown in FIG. 3, the centrifugation 1 unit 76 was used to produce solid and liquid separation of the harvested cell broth from the reactor of fermentation unit 72 and the broth was stored in the optional storage unit 74. The duration was set to be 4 hours. Through centrifugation, 98% (m/m) of solids, which were cells containing PHBV granules, were separated from the cell broth. And approximately 2% (m/m) of cell solids were left in the spent medium. Additionally, 10% (v/v) of cell broth was assumed to be the cell solids slurry, which would then go to downstream PHBV production processes, and 90% (v/v) of cell broth was the supernatant from the centrifugation process, which contained the majority of salts, sugars, and other leftover nutrients from the spent medium.

The output streams of centrifugation 1 unit were: (1) cell mass stream containing PHBV, 31.1 MT/batch; residual biomass, 19.8 MT/batch; Spent MSM, 15.5 MT/batch; glucose, 0.38 MT/batch; galactose, 0.38 MT/batch; lactose, 0.76 MT/batch; ammonium chloride, 0.07 MT/batch; enzyme, 0.05 MT/batch; water, 103.7 MT/batch; (2) spent medium stream containing PHBV, 0.64 MT/batch; residual biomass, 0.36 MT/batch; spent MSM, 139.6 MT/batch; glucose, 3.4 MT/batch; galactose, 3.4 MT/batch; lactose, 6.8 MT/batch; ammonium chloride, 0.7 MT/batch; enzyme, 0.5 MT/batch; water, 587.8 MT/batch. The mass compositions of cell mass stream and spent medium stream were 60.4% and 79.1% respectively.

The cell mass stream from the centrifugation 1 unit 76 proceeded further to downstream processes in extraction unit 80, and the spent medium stream was delivered to an evaporation unit 78 in FIG. 3 or disposed of as high salinity wastewater in the alternative systems. As shown in FIG. 3, the spent medium stream was subjected to an additional unit operation of evaporation 78 to further process the spent saline medium from cell broth for salt recycling. Here, concentrated brine from the evaporation unit 78 was sent to the blending unit 70 for reuse in the fermentation unit 74. Additionally, the water condensate from the evaporator unit 78 can be recycled back to the hydrolysis unit 62.

The evaporation process took 6 hours per batch. It was assumed that 50% (m/m) water was evaporated from the spent medium, and 100% vapor got condensed and the water was reused in the following hydrolysis batch run. After evaporation, the spent medium turned into a brine concentrate stream, where all of the salts from spent medium were reclaimed. The model assumed that 90% of the brine concentrate was reused in the following fermentation batch run, and the leftover 10% was treated as the high saline wastewater.

The concentrated cell mass stream from centrifugation 1 was then fed into a tank to extract PHBV granules from cells in extraction unit 80. A solution of 0.1% (m/m) of Sodium dodecyl chloride (SDS) in water was used as a surfactant to facilitate the extraction process for H. mediterranei. The extraction process in extraction unit 80 was conducted with continuous agitation and heated at 37° C. for 4 h.

After extraction, a 100% vessel working volume with 80.2% (m/m) water was transferred out to the centrifugation 2 unit 82 for a second centrifugation process to concentrate the PHBV extract from the mixed solution in this embodiment. The process duration of centrifugation 2 was set at 2 h. The recovery efficiencies of mass and volume in this process were assumed to be the same as in centrifugation 1. The output raw extract stream consisted of PHBV, 30.5 MT/batch; residual biomass, 2.0 MT/batch; MSM, 1.5 MT/batch; glucose, 0.04 MT/batch; galactose, 0.04 MT/batch; lactose, 0.07 MT/batch; ammonium chloride, 0.007 MT/batch; enzyme, 0.005 MT/batch; and water, 55.1 MT/batch (61.7% m/m). The supernatant from the centrifugation 2 unit 82 was subjected to wastewater treatment with the local sewer price.

The raw extract stream from the centrifugation 2 unit 82 was then processed through a wash run 84, where it was mixed with a 89.3 MT/batch of water to remove most of the soluble compounds and to purify the PHBV extract.

After the wash run 84, the streams were centrifuged again in the centrifugation 3 step in centrifugation unit 86, where the recovery efficiencies of mass and volume in this process were assumed to be the same as the centrifugation 1 and 2 steps. The output purified extract stream contained PHBV, 29.8 MT/batch; residual biomass, 0.2 MT/batch; MSM, 0.2 MT/batch; glucose, 4 kg/batch; galactose, 4 kg/batch; lactose, 8 kg/batch; ammonia chloride, 0.8 kg/batch; enzyme, 0.5 kg/batch; and water, 43.3 MT/batch (59% m/m).

Finally, the purified extract stream was treated by a spray dryer in spray drying unit 88 to yield dry PHBV powder with less than 5% MC, as the final product stream 90 of the system. The dryer was operated at 70° C. for 12 h to achieve a final loss on drying (LOD) of 5%.

### Example 2

To further demonstrate the capabilities of the systems and methods, the mass and energy flows of three system scenarios were compared. Scenario 1 was the system without enzyme reuse or spent salt recycling structures or processes. Scenario 2 was the system with the enzyme reuse but without spent salt recycling structures or processes. Scenario 3 was the system shown schematically in FIG. 3 with both enzyme reuse and spent salt recycling structures and processes. In Scenario 2, the ultrafiltration unit was added right after the hydrolysis tank to concentrate and reuse the enzyme separated from the hydrolysate streams. The ultrafiltration unit may not only save costs required for purchasing new enzymes but may also minimize the influence of enzyme accumulation in the subsequent processes.

Based on the Scenario 2, Scenario 3 added an evaporation process after the centrifugation 1, where the spent saline medium (SSM) was further concentrated to yield a brine concentrate and water condensate. The brine concentrate was then recycled back to the fermentation process, and the water condensate went into the hydrolysis process. The SSM recovery and water reclamation strategies can highly reduce the raw material input and minimize salt discharge to the environment, making this biorefinery system more environmentally friendly.

The model aimed at converting the daily input of feedstock into PHBV dry powder within the same time frame, which can be available to the market. Therefore, the operating schedule was designed to fit the time frame by adopting the staggered operation mode for the main production tanks, which resulted in a faster recipe cycle time of 24 h than the recipe batch time of 123 h. The annual operating time was assumed to be 7899 h in the model, and there were 325 batch runs per year. These values were considered for the calculations of materials and energy flows of the system, and equipment sizing. The mass and energy balance were determined in the model. The mass of the input and output streams of the three scenarios are shown in Table 2.

In Scenario 1, the major input streams were (MT/batch): lactose, 168.7; water, 1005.7; enzyme, 0.5; salt mix, 172.4; nutrient mix, 16.9; air, 9499.5; and SDS, 0.3. The target product stream (PHBV) is 29.8 MT/batch, which corresponds to an overall yield of 18% from lactose input. The major wastewater streams are (MT/batch): SSM, 743.2; and wastewater, 359.6. The wastewater is the sum of normal-salinity wastewater 1 and wastewater 2 streams derived from PHA extraction and wash operations. The SSM is treated as high-salinity wastewater, which costs considerably more than normal-salinity wastewater in terms of treatment and disposal. The vent stream output is 9630.5 MT/batch, which contains air and biogenic CO₂ emitted from various tanks.

Scenarios 2 and 3 have the same input for lactose, nutrient mix, air and SDS, and the same output for PHBV, wastewater and vent, because of the mass balance achieved for the same production target per batch. The input enzyme in Scenario 2 and 3 is 0.1 MT/batch, which is only 20% of that in Scenario 1, since it was assumed that the enzyme was separated from the hydrolysates stream through an ultrafiltration unit and 80% of spent enzyme was reused in the following batch. Additionally, since there is an evaporation unit in the Scenario 3 for salt and water recovery and recycling, the input amounts of salt mix and water are 46.7 and 458.2 MT/batch respectively, which are around 27% and 46% of those materials used in Scenario 1 and 2. The output amount of SSM in Scenario 3 is 44.3 MT/batch, which is only 6% of that in the former two scenarios. The reductions of input and output materials in Scenario 3 can lead to economic benefits of the production system.

According to the energy balance conducted in the model, the annual amounts of utilities consumed in Scenario 1 include 1.64E+8 kW-h of electricity, 2.8E+4 MT of steam, and 1.96E+7 MT of cooling water. Scenario 2 has the same consumptions of utilities to Scenario 1. The utility of steam in Scenario 3 is 1.4E+5 MT, which is about 5 times of that in the other two scenarios. This high consumption of steam is due to the additional evaporation unit in Scenario 3.

The material and energy balances of the model were also used to determine the size and the operational throughput of each equipment. The vessel volumes of the tanks are estimated based on the volumes of input and output streams and the assumption that 85% of vessel volume is working volume and the left 15% is used as headspace to prevent pressure build up. The throughputs of centrifuges are adjusted in a way that the total processing time of the three centrifuges is less than the cycle time (24 h), so that the downstream processes of PHA extraction, purification and drying can be completely within the same batch time. The unit costs of tanks were estimated based on a rate of 793$/m³.

### Example 3

A techno-economic analysis was conducted on the PHA production system by using cheese processing by-product streams as feedstock. The three scenarios with different unit operations were compared for materials and energy flows, major cost items in the direct capital cost and annual operating cost, and sensitivity analysis. The important factors of profitability including equipment cost (EC), direct capital cost (DCC) and annual operating cost (AOC) have been compared among the three scenarios.

A review of the AOC of the three scenarios revealed that the raw materials cost (RMC) is the largest share of the AOC in all scenarios, which is consistent with previous findings from existing PHA production systems. The results of the RMC breakdown indicate that the prices and input mass of raw materials, particularly feedstocks, are the major factors that can influence the overall economics of the PHA production system. It was observed that the largest portion of RMC is the cost of lactose feedstock, which accounts for 49% of RMC in Scenario 1, 63% in Scenario 2, and 76% in Scenario 3. The lactose cost depends on the input mass and the lactose price.

The cost of enzyme is the second largest share in Scenario 1, which accounts for 28% of RMC. However, it is reduced by 86% in Scenario 2 and 3, which is a result of enzyme recycling. In the case of high enzyme costs, the use of technologies to recycle enzyme in the hydrolysis step of the production system may be necessary to benefit the overall economics of the system.

High salt is advantageous in helping eliminate the energy-intensive pasteurization or sterilization operations. However, the costs required to purchase salts and treat/dispose of high-saline wastewater is an issue for this type of production system. Therefore, the recycling of SSM using an evaporation-condensation process may be necessary to obtain the financial and environmental benefits. The lower RMC costs observed in scenarios 2 and 3 are due to the savings of enzyme, salt mix and water through enzyme reuse and SSM recycling.

The cost of utilities is the second largest share of AOC. Due to the additional unit operations in Scenario 2 and 3, the energy consumption, EC and DCC are higher than Scenario 1. However, since those additional units lead to savings of major inputs including salt mix, water, enzyme, and output of costly wastewater, the latter two scenarios, particularly Scenario 3, have less AOC than Scenario 1.

Given that feedstock cost is found to be the largest portion of RMC, the breakeven price of PHA is equally sensitive to the changing lactose price in all scenarios. The breakeven price was found to be less sensitive to lactase enzyme price than lactose price, and enzyme recycling strategy may not be economically beneficial with a cheaper enzyme price. Scenario 3 is the most profitable case among all others, and the use of DLP as feedstock results in the lowest breakeven price which can be less than 4 $/kg PHA. The low breakeven price enables PHA to be economically competitive with conventional plastics and common bioplastics. This can be beneficial to the dairy industry by adding an additional revenue stream to dairy by-products. The case of making PHA from cheese by-products can be applied to other dairy by-products and waste streams. Therefore, PHA production from dairy derived by-products has the potential to grow dairy markets into non-food products, which also offers a profitable business case for the bioplastic industry.

### Example 4

The capability of recycling salt in the PHA production system that uses halophilic microbes is an important element to reduce costs and the environmental impact from the disposal of spent saline media. One effective and efficient approach for direct recycling and reuse of spent saline medium during the processes of cell cultivation and PHA production by Haloferax mediterranei was demonstrated.

The pure-strain Haloferax mediterranei (ATCC 33500) was used for cell cultivation and PHA production throughout the study. The enzymatic hydrolysate of lactose (whey sugars) was loaded to provide a soluble COD of 20 g/L. The saline medium had a salt content of around 19%. Nitrogen and phosphorous sources were added to lactose hydrolysate with 2.8 g/L NH₄Cl and 0.7 g/L KH₂PO₄. The 250-mL bioreactors with a working volume of 200 mL were used for cell cultivation experiments. The bioreactors were housed in an incubator with a controlled temperature at 37 degrees Celsius. The pH of cell broth was maintained around 7 by adding 30 mM NaHCO₃ as buffer. Active seed of H. mediterranei was loaded to the culture medium to give an initial optical density (OD) of 1.0. Air was provided to the bioreactors through air pumps and humidifiers.

Four consecutive batch runs were conducted with the spent salts being recycled and reused for cell cultivation. At the end of each batch run, the cell mass was separated from spent medium through centrifugation at 8000 rpm for 30 min. Prior to the next batch, around 20% volume of spent saline medium was sampled for analysis, and 80% volume of spent saline medium was processed through rotary evaporation. The evaporation was conducted with applied vacuum and a temperature of 57° C. for around 30 mins. The evaporation treatment was stopped exactly when salt crystals started to appear in the brine. The evaporation process removed approximately 50% water. The remaining brine solution contained all the salts from the spent medium input. To prepare the culturing medium for the next batch, 20% salts were added to the spent brine solution to compensate for the losses of salts from sampling. The same loadings of lactose hydrolysate, nitrogen and phosphorous were added to the culturing medium. Water was also added to increase the medium volume to 200 mL. The culturing medium was used for cell cultivation in the following batch run.

Cell growth curves of the four batch runs were plotted with the direct recycling of spent saline medium after water removal by evaporation. All batch runs had similar cell growth rates and the cultivation times (from 120 h to 144 h) to reach the stationary growth phase. The observed final cell density was between an optical density (OD) of 10 to 12 for all batches.

The production of cell dry mass and PHA were observed to be 3 to 5.5 g/L and 1.5 to 2 g/L, which were similar in all batches and aligned well with the results of cell growth curves. The HB and HV contents of the PHBV produced from different batches with recycled saline media were evaluated. It was found that the consecutive recycled batches gave stable HB and HV contents, which were around 80% and 20% of PHBV, respectively. The results suggested the production and quality of the PHBV remained stable with the direct recycling of the spent saline medium.

### Example 5

To further demonstrate the capability of recycling salt media in the PHA production system, an alternative approach for recycling of spent saline media with chemical treatment using an oxidizing agent, such as hydrogen peroxide (H₂O₂) was demonstrated. In the circumstances where the saline medium contains a substantial amount of organic matter, treatment of spent saline medium with oxidizing chemicals such as H₂O₂ or NaClO may reduce the residual matter and improve the quality of the recycled medium.

To demonstrate the approach, pure-strain Haloferax mediterranei (ATCC 33500) was used for cell cultivation and PHA production. A fermented food waste permeate was used as the substrate to provide a soluble COD of 20 g/L. The saline medium had a salt content of around 19%. The 250-mL bioreactors with a working volume of 200 mL were used for cell cultivation experiments. The bioreactors were housed in an incubator with a controlled temperature at 37 degrees Celsius. The pH of cell broth was maintained around 7 by adding around 30 mM NaHCO₃ as buffer. Active seed of H. mediterranei was loaded to the culture medium to give an initial optical density (OD) of 1.0. The bioreactors were aerated using moist air that was provided through air pumps and humidifiers.

A total of three consecutive batch runs were conducted with the spent salts being recycled and reused for cell cultivation. At the end of each batch run, the cell mass was separated from spent medium through centrifugation at 8000 rpm for 30 min. Prior to the next batch, around 20% volume of spent saline medium was sampled for analysis, and 80% volume of spent saline medium was processed through rotary evaporation. The evaporation was conducted with applied vacuum and a temperature of 57 degrees Celsius for around 30 mins. The evaporation treatment was stopped exactly when salt crystals started to appear in the brine. The evaporation process removed 50% water. And the remaining brine solution was about 50% of the original spent saline medium and was then used for cell cultivation in the following batch run.

Due to the presence of a substantial amount of organic matter in spent saline media (over 12 g/L COD), the cells failed to grow well in the second batch. Thereafter, the saline media was treated with chemicals in order to reduce the COD and remove the inhibitory compounds. The spent saline medium of second batch was treated with H₂O₂ by adding aqueous solution containing 50% H₂O₂ to the spend saline medium at 5% of the total volume. After treatment, the spent saline medium was subjected to the same evaporation process as mentioned previously to generate a brine solution, which was reused for the following batch. The same conditions and nutrients for cell cultivation were provided to all batch runs.

The mass balance of the salts in the four batch runs were tabulated. The results suggested that around 80% spent salts were recycled and reused in the second through forth batches, with 20% new salts added to each batch maintained a stable salt content of the medium.

Cell growth curves were plotted of the three batch runs with the recycling of spent saline medium after water removal by evaporation, where the second batch of recycled saline medium was without treatment and the third batch of recycled the saline media was treated with H₂O₂ for comparison. It was found that the cell growth of the second batch was unsatisfactory. However, after the H₂O₂ treatment of the spent saline medium, the cell growth of the third batch was good and substantially better than the cell growth of the first batch. The final cell density of the third batch was around an optical density of 12. The color of the medium changed from brown (the natural color of fermented food waste permeates) to light yellow and the medium became clear after the H₂O₂ treatment.

From the description herein, it will be appreciated that the present disclosure encompasses multiple implementations which include, but are not limited to, the following:

A method for producing polyhydroxyalkanoates (PHA) from organic waste, the method comprising: (a) preparing a support media with one or more strains of saline tolerant halophilic microorganisms; (b) adding a volume of decomposed organic waste to the support media; (c) growing the halophilic microorganisms in the media; and (d) extracting PHA from the halophilic microorganisms collected from the media.

The method of any preceding or following implementation, wherein the decomposed organic waste comprises one or more of volatile fatty acids, lactic acid, sugars, and other nutrients.

The method of any preceding or following implementation, wherein the organic waste is decomposed by anerobic fermentation.

The method of any preceding or following implementation, wherein the organic waste is decomposed by hydrolysis selected from the group of thermal, chemical and enzymatic hydrolysis.

The method of any preceding or following implementation, further comprising: separating solids from the decomposed organic waste to produce an aqueous solution of nutrients.

The method of any preceding or following implementation, further comprising: extracting water from the solution of nutrients to concentrate nutrients from the solution; and recycling extracted water from the solution of nutrients.

The method of any preceding or following implementation, wherein the decomposed organic waste added to the support media comprises a solution of the concentrated nutrients.

The method of any preceding or following implementation, wherein the strains of saline tolerant halophilic microorganisms is a microorganism selected from the group consisting of Haloferax, Halomonas, Haloarcula, Halococcus, Halobacterium and Natrinema microorganisms.

The method of any preceding or following implementation, wherein the support medium for the halophilic microorganisms comprises a salt concentration within the range of 5% to 30% (w/v).

A method for producing polyhydroxyalkanoates (PHA) from organic waste, the method comprising: (a) decomposing organic waste into an aqueous solution of nutrients; (b) removing solids from the aqueous solution to obtain a particle-free aqueous solution of nutrients; (c) removing water from the particle-free aqueous solution to obtain a concentrated nutrient solution; (d) mixing the concentrated nutrient solution with halophilic microorganisms in a saline solution to produce a saline growth media; (e) growing the halophilic microorganisms in the saline growth media to produce a cell biomass of halophilic microorganisms that have synthesized PHA intracellularly; (f) harvesting the cell biomass from the media; and (g) extracting PHA from the cell biomass.

The method of any preceding or following implementation, wherein the organic waste is decomposed by anerobic fermentation.

The method of any preceding or following implementation, wherein the organic waste is decomposed by hydrolysis selected from the group of thermal, chemical and enzymatic hydrolysis.

The method of any preceding or following implementation, further comprising: recycling water removed from the particle-free aqueous solution to obtain a concentrated nutrient solution for organic waste decomposition.

The method of any preceding or following implementation, wherein the strains of saline tolerant halophilic microorganisms is a microorganism selected from the group consisting of Haloferax, Halomonas, Haloarcula, Halococcus, Halobacterium and Natrinema microorganisms.

The method of any preceding or following implementation, wherein the saline growth media for the halophilic microorganisms comprises a salt concentration within the range of 5% to 30% (w/v).

The method of any preceding or following implementation, further comprising: recycling the saline growth media after cell biomass removal; and adding halophilic microorganisms to the recycled saline growth media.

A method for producing polyhydroxyalkanoates (PHA) from organic waste, the method comprising: (a) hydrolyzing organic waste to produce an aqueous solution of hydrolysates; (b) filtering the aqueous solution of hydrolysates; (c) blending the filtrate of the filtered solution of hydrolysates with a saline culture media and halophilic microbes that synthesize PHA intracellularly; (d) incubating the blended culture media and halophilic microbes; (e) separating the incubated microbes from the blended saline culture media; (f) recycling saline culture media; and (g) extracting PHA from the separated incubated microbes.

The method of any preceding or following implementation, further comprising: hydrolyzing the organic waste with an enzyme; and recycling enzymes from the filtrate for further hydrolysis of organic waste.

The method of any preceding or following implementation, further comprising: extracting PHA from the separated incubated microbes with a surfactant.

The method of any preceding or following implementation, further comprising: applying a surfactant to the separated incubated microbes to produce an extraction mix; centrifuging the extraction mix to produce a raw extract; washing the raw extract with water; centrifuging the washed extract to produce a washed extract; and drying the washed extract to produce a PHA powder final product.

The method of any preceding or following implementation, further comprising: treating saline culture media after microbe separation with an oxidizing chemical; and recycling the treated saline culture media to the blending step.

A method for producing polyhydroxyalkanoates (PHA) from organic waste, the method comprising: (a) an organic waste decomposition step where organic waste is converted into soluble nutrients such as volatile fatty acids (VFA), lactic acid, sugars, nitrogen and phosphorous, and wherein decomposition is achieved by anaerobic fermentation, hydrolysis by thermal, chemical and enzymatic treatments, or other methods; (b) a solid liquid separation step to obtain a particle-free aqueous solution containing nutrients; (c) a nutrient concentration step to remove most of water and obtain a highly concentrated nutrient solution; (d) a PHA production step where the nutrient concentrates are utilized by halophilic microorganisms in a saline nutrient solution to synthesize PHA intracellularly; (e) a cell biomass harvest step which yields cell biomass and recycles saline nutrient solution back to the PHA production step; and (f) a PHA extraction step where PHA is extracted from cell biomass and becomes the final product.

The method of any preceding or following implementation, wherein the oxidizing chemical comprises H₂O₂ or NaClO.

As used herein, term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly the reader should consult the application as filed for the original content of the disclosure. Any deletion of content of the disclosure should not be construed as a disclaimer, forfeiture, or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Nutrient Levels of Liquid Broth from Anaerobic
Fermentation of Food Waste

| | | Average value | |
|---|---|---|---|
| Components | Unit | (standard deviation) | |
| pH | — | 5.06 | (0.01) |
| Soluble COD | g/L | 21.17 | (1.60) |
| Nitrogen (total) | mg/L | 24 | |
| Phosphorous (total) | mg/L | 38 | (1) |
| Ammonia-N | mg/L | 20 | (4) |
| VFA (total) | g/L | 11.22 | (0.01) |
| Acetic acid | g/L | 3.98 | (0.14) |
| Propionic acid | g/L | 1.40 | (0.61) |
| Iso-butyric acid | g/L | 0.37 | (0.25) |
| Butyric acid | g/L | 5.15 | (0.57) |
| Iso-valeric acid | g/L | 0.24 | (0.10) |
| Valeric acid | g/L | 0.08 | (0.04) |
| Lactic acid | g/L | 0.10 | (0.03) |

TABLE 2
The mass of input and output streams of Scenarios 1 through 3

| Input | Sce- | Sce- | Sce- | Output | Sce- | Sce- | Sce- |
|---|---|---|---|---|---|---|---|
| (MT/ | nario | nario | nario | (MT/ | nario | nario | nario |
| batch) | 1 | 2 | 3 | batch) | 1 | 2 | 3 |
| Water | 1005.7 | 1005.7 | 458.2 | PHBV | 29.8 | 29.8 | 29.8 |
| Lactose | 168.7 | 168.7 | 168.7 | SSM | 743.2 | 743.2 | 44.3 |
| Enzyme | 0.5 | 0.1 | 0.1 | Waste- | 359.6 | 359.6 | 359.6 |
| | | | | water | | | |
| Salt | 172.4 | 172.4 | 46.7 | Vent | 9630.5 | 9630.5 | 9630.5 |
| mix | | | | | | | |
| Nutrient | 16.9 | 16.9 | 16.9 | | | | |
| mix | | | | | | | |
| Air | 9499.5 | 9499.5 | 9499.5 | | | | |
| SDS | 0.3 | 0.3 | 0.3 | | | | |

Scenario 1: PHA production base model;
Scenario 2: PHA production model with enzyme reuse;
Scenario 3: PHA production model with enzyme reuse and spent brine recycling.

What is claimed is:

1. A method for producing polyhydroxyalkanoates (PHA) from organic waste, the method comprising:
(a) preparing a support media with one or more strains of saline tolerant halophilic microorganisms;
(b) adding a volume of decomposed organic waste to said support media;
(c) growing the halophilic microorganisms in the media; and
(d) extracting PHA from said halophilic microorganisms collected from the media.

2. The method of claim 1, wherein the decomposed organic waste comprises one or more of volatile fatty acids, lactic acid, sugars, and other nutrients.

3. The method of claim 1, wherein said organic waste is decomposed by anerobic fermentation.

4. The method of claim 1, wherein said organic waste is decomposed by hydrolysis selected from the group of thermal, chemical and enzymatic hydrolysis.

5. The method of claim 1, further comprising:
separating solids from said decomposed organic waste to produce an aqueous solution of nutrients.

6. The method of claim 1, further comprising:
extracting water from said solution of nutrients to concentrate nutrients from the solution; and
recycling extracted water from said solution of nutrients.

7. The method of claim 6, wherein said decomposed organic waste added to said support media comprises a solution of said concentrated nutrients.

8. The method of claim 1, wherein said strains of saline tolerant halophilic microorganisms is a microorganism selected from the group consisting of Haloferax, Halomonas, Haloarcula, Halococcus, Halobacterium and Natrinema microorganisms.

9. The method of claim 1, wherein said support medium for said halophilic microorganisms comprises a salt concentration within the range of 5% to 30% (w/v).

10. A method for producing polyhydroxyalkanoates (PHA) from organic waste, the method comprising:
(a) decomposing organic waste into an aqueous solution of nutrients;
(b) removing solids from the aqueous solution to obtain a particle-free aqueous solution of nutrients;
(c) removing water from the particle-free aqueous solution to obtain a concentrated nutrient solution;
(d) mixing the concentrated nutrient solution with halophilic microorganisms in a saline solution to produce a saline growth media;
(e) growing the halophilic microorganisms in the saline growth media to produce a cell biomass of halophilic microorganisms that have synthesized PHA intracellularly;
(f) harvesting the cell biomass from the media; and
(g) extracting PHA from the cell biomass.

11. The method of claim 10, wherein said organic waste is decomposed by anerobic fermentation.

12. The method of claim 10, wherein said organic waste is decomposed by hydrolysis selected from the group of thermal, chemical and enzymatic hydrolysis.

13. The method of claim 10, further comprising:
recycling water removed from the particle-free aqueous solution to obtain a concentrated nutrient solution for organic waste decomposition.

14. The method of claim 10, wherein said strains of saline tolerant halophilic microorganisms is a microorganism selected from the group consisting of Haloferax, Halomonas, Haloarcula, Halococcus, Halobacterium and Natrinema microorganisms.

15. The method of claim 10, wherein said saline growth media for said halophilic microorganisms comprises a salt concentration within the range of 5% to 30% (w/v).

16. The method of claim 10, further comprising:
recycling the saline growth media after cell biomass removal; and
adding halophilic microorganisms to the recycled saline growth media.

17. A method for producing polyhydroxyalkanoates (PHA) from organic waste, the method comprising:
(a) hydrolyzing organic waste to produce an aqueous solution of hydrolysates;
(b) filtering the aqueous solution of hydrolysates;
(c) blending the filtrate of the filtered solution of hydrolysates with a saline culture media and halophilic microbes that synthesize PHA intracellularly;
(d) incubating the blended culture media and halophilic microbes;
(e) separating the incubated microbes from the blended saline culture media;
(f) recycling saline culture media; and
(g) extracting PHA from the separated incubated microbes.

18. The method of claim 17, further comprising:
hydrolyzing the organic waste with an enzyme; and
recycling enzymes from the filtrate for further hydrolysis of organic waste.

19. The method of claim 17, further comprising:
extracting PHA from the separated incubated microbes with a surfactant.

20. The method of claim 19, further comprising:
applying a surfactant to the separated incubated microbes to produce an extraction mix;
centrifuging the extraction mix to produce a raw extract;
washing the raw extract with water;
centrifuging the washed extract to produce a washed extract; and
spray drying the washed extract to produce a PHA powder final product.

21. The method of claim 17, further comprising:
treating saline culture media after microbe separation with an oxidizing chemical; and
recycling the treated saline culture media to the said blending step.

22. The method of claim 21, wherein said oxidizing chemical comprises H₂O₂ or NaClO.
